(19) United States
(12) Patent Application Publication
(10) Pub. No.: US 2025/0276303 A1
(43) Pub. Date: Sep. 4, 2025

(54) Supported TiOx Core-Shell Catalyst and Preparation Method and Application Thereof

(71) Applicant: TIANJIN UNIVERSITY, Tianjin (CN)

(72) Inventors: Jinlong GONG, Tianjin (CN); Sai CHEN, Tianjin (CN); Yiyi XU, Tianjin (CN); Chunlei PEI, Tianjin (CN); Donglong FU, Tianjin (CN)

(21) Appl. No.: 18/893,426

(22) Filed: Sep. 23, 2024

(30) Foreign Application Priority Data
Feb. 29, 2024 (CN) 2024102264332

(51) Int. Cl. B01J 21/06; B01J 23/755; B01J 23/76; B01J 35/30; B01J 35/53; B01J 37/02; B01J 37/04; B01J 37/12; C07C 5/32

(52) U.S. Cl. CPC B01J 21/063; B01J 23/755; B01J 23/76; B01J 35/398; B01J 35/53; B01J 37/0209; B01J 37/04; B01J 37/12; C07C 5/325

(57) ABSTRACT

The present disclosure discloses a supported TiOₓ core-shell catalyst and a preparation method and application thereof. An Al₂O₃ support is loaded with a Ni@TiOₓ core-shell structure, and the core-shell structure includes a metal Ni core and a TiOₓ shell. The preparation method includes the steps of firstly, adding aluminum alkoxide, an organotitanium compound, and a surfactant to isopropanol solvent and stirring them to be mixed well, and then dropwise adding dilute nitric acid to be hydrolyzed completely; aging obtained sol at room temperature, and completely drying it under vacuum; then calcining the obtained solid step by step; impregnating the solid in a Ni(NO₃)₃·6H₂O solution to be completely dried after being ultrasonically dispersed well; and finally calcining and then reducing the obtained solid, to obtain the Al₂O₃ supported Ni@TiOₓ core-shell catalyst.

### CROSS-REFERENCE TO RELATED APPLICATION

This application claims priority from the Chinese patent application 2024102264332 filed Feb. 29, 2024, the content of which is incorporated herein in the entirety by reference.

### TECHNICAL FIELD

The present disclosure belongs to the technical field of supported catalysts, and particularly relates to a supported TiOₓ core-shell catalyst and a preparation method and application thereof.

### BACKGROUND ART

Propylene is one of the important basic chemical raw materials for industrial production, which can be used to produce different downstream chemical products according to its content. Refinery grade propylene (50%-70%) is suitable for the production of isopropanol or isopropylbenzene or the like; chemical grade propylene (92%-96%) is suitable for the production of oxo-alcohols, acrylonitrile, acrylic acid, propylenoxide, isopropanol, isopropylbenzene or the like, and polymeric grade propylene (greater than 99.5%) can be used to produce polypropylene, ethylene-propylene rubber, propylene chloride or the like. These products are widely applied to a construction material industry, a textile industry, an automobile industry or other industries due to their excellent properties. In recent years, the global demand for propylene has increased greatly, which has promoted the rapid growth of its production capacity, and it is predicted that the demand for propylene will grow at an average annual growth rate of 2%-3% until 2035. At present, the supply of propylene mainly derives from by-products of steam cracking and fluid catalytic cracking (FCC) processes of petroleum hydrocarbons. However, in recent years, with the rapid development of a hydraulic fracturing technology, shale gas condensate (NGLS) rich in methane, ethane, propane and other low-carbon alkane can be extracted and exploited on a large scale at a relatively low cost, resulting in a substantial increase in propane yield. Due to the low cost of shale gas, the raw material for the steam cracking process, which mainly produces ethylene and by-produces propylene, has also shifted from naphtha to ethane based on shale gas, and most of the steam cracking devices have been dismantled or transformed into an ethane cracking device. The supply of propylene is further reduced, and the price of propylene rises sharply. The combination of the two factors has led to the continuous increase in propylene-propane price difference since 2016, and the propane dehydrogenation technology has significantly increased its production capacity due to its cost advantage, becoming a more economical option.

A reaction formula of propane dehydrogenation is: C₃H₈⇄C₃H₆+H₂, ΔH_{298K}=124.3 kJ/g·mol. The reaction is an equilibrium reaction with strong heat absorption and increased molecular number. The thermodynamics is limited, and the conditions of high temperature and low partial pressure of propane are favorable for the reaction. Supported CrOₓ and Pt catalysts are two important industrial catalysts, which are used in production processes where propane dehydrogenation has been industrialized, namely Catofin process from Lummus Co. and Oleflex process from UOP Co., the CrOₓ catalyst used in the Catofin process is plagued by carbon deposition inactivation, requiring frequent regeneration treatment, and CrOₓ will also cause serious pollution to the environment. The Pt catalyst used in the Oleflex process has excellent ability to activate C—H bonds of alkane, however, the application of Pt as a precious metal is greatly limited by its high price. In conclusion, cheap and environment-friendly alternative catalysts have attracted widespread attention.

Oxides such as ZrO₂, TiO₂, and WO₃ have long been regarded as having no catalytic activity for propane dehydrogenation. However, some studies in recent years have shown that the catalytic activity increases after stoichiometric oxide surfaces are partially reduced to form oxygen vacancies and coordinatively unsaturated metal cation sites. It is shown that these oxides also have potential activity, among which TiO₂ is a potential catalyst since it is abundant in reserve, cheap and easy to obtain. At present, the problem is that the intrinsic activity of TiO₂ as an active species is still low, so it has no industrial application value of catalysts.

### SUMMARY

The present disclosure provides a supported TiOₓ core-shell structural catalyst and a preparation method and application thereof to solve the technical problems of low catalytic activity of existing TiO₂-based catalysts. The catalyst has high activity comparable to industrial precious metal catalysts, and high selectivity and stability, breaks through the limitation that the activity of oxide-based catalysts is low and does not meet the requirements of industrial application, and can be applied as a catalyst in preparation of olefin from dehydrogenation of light alkane.

To solve the above technical problems, the present disclosure is achieved by the following technical solutions:

In an aspect of the present disclosure, a supported TiOₓ core-shell catalyst is provided, which adopts Al₂O₃ as a support, the Al₂O₃ support is loaded with a Ni@TiOx core-shell structure, the Ni@TiOx core-shell structure includes a metal Ni core and a TiOₓ (1<x<2) shell; and a molecular formula of the catalyst is denoted as NimTin/Al₂O₃, wherein m:n=1:(1-6).

Furthermore, a mass percentage of TiOₓ is 5%-15% based on a mass of the Al₂O₃ support.

Furthermore, m:n=1:4.

According to another aspect of the present disclosure, a preparation method of the above supported TiOₓ core-shell catalyst is provided, including the following steps:
(1) adding aluminum alkoxide, an organotitanium compound, and a surfactant to isopropanol solvent and stirring them to be mixed well;
(2) dropwise adding dilute nitric acid to the mixed solution obtained in step (1) to be hydrolyzed completely;
(3) aging sol obtained in step (2) at room temperature, and completely drying the sol under vacuum;
(4) calcining the solid obtained in step (3) step by step;
(5) impregnating the solid obtained in step (4) in Ni(NO₃)₃·6H₂O solution to be completely dried after being ultrasonically dispersed well; and
(6) calcining the solid obtained in step (5), and then reducing the solid at 400-700° C., to obtain the Al₂O₃ supported Ni@TiOₓ core-shell catalyst.

Furthermore, in step (1), the aluminum alkoxide is one of aluminum tri-sec-butoxide (ATSB) and aluminum isopropoxide (Al(Opri)₃); the organotitanium compound is one of tetrabutyl titanate (TTB) and isopropyl titanate (TTP); the surfactant is one of cetyl trimethyl ammonium bromide (CTAB) and cetyltrimethylammonium chloride (CTAC); and the organic alcohol solvent is isopropanol or ethanol.

Furthermore, in step (3), the vacuum drying is to dry in a vacuum oven at 60-80° C. for 18-24 hours.

Furthermore, in step (4), the calcining step by step is to firstly calcine at 200-300° C. for 2-3 hours, and then ramp up to 500-600° C. for calcining for 3-4 hours.

Furthermore, in step (6), the calcining temperature is 500-600° C., and calcining time is 2-4 hours; and reducing time is 1-2 hours.

According to yet another aspect of the present disclosure, an application of the above supported TiOₓ core-shell catalyst in preparation of olefin from dehydrogenation of light alkane is provided.

Furthermore, the light alkane is propane, and the olefin is propylene.

The present disclosure has the following beneficial effects:

The supported TiOₓ core-shell catalyst of the present disclosure adopts cheap and easily available non-precious metal oxide TiOₓ as an active component, has high activity comparable to precious metal Pt series catalysts commonly used in industry, but its cost is greatly reduced; and the coordination environments and electron properties of the active site TiOₓ are modulated by the interaction between sub-surface metal Ni as an electron promoter and the surface TiOₓ, so as to accelerate C—H bond activation and H₂ desorption in the dehydrogenation reaction process, thereby significantly improving the intrinsic catalytic activity of TiOₓ. It is proved through various characterization techniques that due to coverage of Ni sites by the TiOₓ overlayer when increasing reduction temperature, adverse effects of Ni sites with high C—C bond cleavage activity on dehydrogenation selectivity are avoided, assuring high selectivity of the TiOₓ-based dehydrogenation catalyst.

The catalyst of the present disclosure is prepared through a sol-gel method and a co-impregnation method, the raw materials are easy to obtain, the process is simple, the repeatability is high, possessing promising industrialized potential.

The catalyst of the present disclosure has a good catalytic effect on preparation of olefin from dehydrogenation of light alkane, the conversion rate of the light alkane at high temperature can reach 40% or above, the olefin selectivity can reach 93% or above, the propylene yield based on the mass of the catalyst can reach about 16.70 mmol·g_{cat}⁻¹·h⁻¹, comparable to industrial catalysts, and breaks through the limitation of oxide-based catalysts' activity which does not meet the requirements of industrial application.

### BRIEF DESCRIPTION OF THE DRAWINGS

FIGS. 1A-B are diagrams showing catalytic performance of catalysts prepared in Examples 1-6, wherein FIG. 1A is a curve of propane conversion over time, and FIG. 1B is a curve of propylene selectivity over time;

FIG. 2 is a diagram showing comparison between catalytic performance of catalysts prepared in Examples 1 and 16;

FIG. 3 is a diagram showing catalytic performance of catalysts prepared in Examples 1, 7, and 8;

FIG. 4 is a diagram showing long-term regeneration stability of the catalyst prepared in Example 1;

FIG. 5 is spherical aberration-scanning transmission electron microscope images of catalysts prepared in Examples 1, 11, and 12 during in-situ reduction;

FIG. 6 is the electron energy loss spectral line scanning profile corresponding to the spherical aberration-scanning transmission electron microscope image of the catalyst prepared in Example 1 during in-situ reduction;

FIG. 7 is a diagram showing comparison among CO adsorption infrared spectroscopy of catalysts prepared in Examples 1, 11, 13, and 14 during in-situ reduction;

FIGS. 8A-B are diagrams showing comparison of in-situ XPS results of catalysts prepared in Examples 1 and 5 under successive gas treatment, wherein FIG. 8A and FIG. 8B respectively correspond to the catalysts prepared in Examples 5 and 1;

FIG. 9 is a diagram showing comparison of EPR results of catalysts prepared in Examples 1 and 5;

FIG. 10 is a diagram showing comparison of curve fitting results of Ti K-edge extended X ray absorption fine structures of catalysts prepared in Examples 1 and 5;

FIGS. 11A-B are diagrams showing propane-temperature programmed surface reaction results of catalysts prepared in Examples 1 and 5, wherein FIG. 11A and FIG. 11B respectively correspond to the catalysts prepared in Examples 5 and 1.

### DETAILED DESCRIPTION OF THE EMBODIMENTS

The present disclosure provides a supported TiOₓ core-shell catalyst, which adopts Al₂O₃ as a support loaded with a Ni@TiOₓ core-shell structure, the Ni@TiOₓ core-shell structure includes a metal Ni core and a TiOₓ (1<x<2) shell; and a molecular formula of the catalyst is denoted as NimTin/Al₂O₃, wherein m:n=1:(1-6), most preferably, m:n=1:4.

In a preferred example of the present disclosure, a mass percentage of TiOₓ is 5%-15% based on a mass of Al₂O₃ support.

The present disclosure further provides a preparation method of a supported TiOₓ core-shell catalyst, including the following steps:
Step (1): aluminum alkoxide, an organotitanium compound, and a surfactant are added to isopropanol solvent and they are stirred to be mixed well.

In a preferred example of the present disclosure, the aluminum alkoxide is one of aluminum tri-sec-butoxide (ATSB) and aluminum isopropoxide (Al(Opri)₃);
in a preferred example of the present disclosure, the organotitanium compound is one of tetrabutyl titanate (TTB) and isopropyl titanate (TTP);
in a preferred example of the present disclosure, the surfactant is one of cetyl trimethyl ammonium bromide (CTAB) and cetyltrimethylammonium chloride (CTAC); and
in a preferred example of the present disclosure, the organic alcohol solvent is isopropanol or ethanol.
Step (2): dilute nitric acid is dropwise added to the mixed solution obtained in step (1) to be hydrolyzed completely.

In some examples of the present disclosure, hydrolysis time is 1 hour.
Step (3): the sol obtained in step (2) is aged at a room temperature, and the sol is completely dried under vacuum.

In a preferred example of the present disclosure, the complete drying under vacuum is to dry in a vacuum oven at 60-80° C. for 18-24 hours.

In a preferred example of the present disclosure, aging time is 24 hours.
Step (4): the solid obtained in step (3) is calcined step by step.

In a preferred example of the present disclosure, the calcining step by step is to firstly calcine at 200-300° C. for 2-3 hours, and then ramp up to 500-600° C. for calcining for 3-4 hours.
Step (5): the solid obtained in step (4) is impregnated in Ni(NO₃)₃·6H₂O solution to be completely dried after being ultrasonically dispersed well.
Step (6): the solid obtained in step (5) is calcined, and then reduced at 400-700° C., to obtain the Al₂O₃ supported Ni@TiOₓ core-shell catalyst.

In a preferred example of the present disclosure, the calcining temperature is 500-600° C., and calcining time is 2-4 hours.

In a preferred example of the present disclosure, the reducing time is 1-2 hours.

The present disclosure further provides an application of the above supported TiOₓ core-shell catalyst in preparation of olefin from dehydrogenation of light alkane, especially an application in preparation of propylene from propane dehydrogenation.

The present disclosure is further described in detail below through specific examples, and the following examples can allow those skilled in the art to understand the present disclosure more comprehensively, instead of limiting it in any manner.

### Example 1

Step (1): 2.174 parts by mass of aluminum tri-sec-butoxide (ATSB), 0.425 part by mass of tetrabutyl titanate (TTB), and 0.182 part by mass of surfactant cetyl trimethyl ammonium bromide (CTAB) are added to isopropanol solvent and stirred for 1.5 hours to be mixed well;
Step (2): mixed solution obtained in step (1) is dropwise added to 3.726 parts by mass of dilute nitric acid to be hydrolyzed for 1 hour;
Step (3): sol obtained in step (2) is aged at a room temperature for 24 hours and then dried in a vacuum oven at 60-80° C. for 18-24 hours;
Step (4): the solid obtained in step (3) is calcined at 200-300° C. step by step for 2-3 hours and then ramped up the temperature to 500-600° C. for calcining for 3-4 hours;
Step (5): 0.09 part by mass of Ni(NO₃)₃·6H₂O is dissolved in 1 mL of deionized water;
Step (6): 1 part by mass of the solid obtained in step (4) is impregnated in the solution obtained in step (5) for ultrasonic treatment for 0.5-1 hour, then naturally dried at room temperature for 12 hours, and then completely dried at 80-100° C.;
Step (7): the solid obtained in step (6) is calcined under air atmosphere at 600° C. for 3 hours and then reduced at 600° C. for 1 hour, to obtain the Al₂O₃ supported Ni@TiOₓ core-shell catalyst, wherein the mass percentage of TiOₓ is 10% based on the mass of the support, and the molecular formula is denoted as Ni1Ti4/Al₂O₃;
Step (8): the prepared Al₂O₃ supported Ni@TiOₓ core-shell catalyst is pressed into pallets and sieved to granular catalysts at 20-40 meshes;
Step (9): the Al₂O₃ supported Ni@TiOₓ granular catalyst is put into a fixed bed reactor, and reaction gas is introduced for reaction, wherein the molar ratio of hydrogen to propane is 1:1, the mass space velocity of propane is 3 h⁻¹, and the balance gas is nitrogen;
Step (10): the spent Al₂O₃ supported Ni@TiOₓ core-shell catalyst is regenerated, air is introduced at 500-550° C. for regeneration for 0.5 hours, and then ramping up to 600° C. for reduction for 1 hour, to obtain the regenerated Al₂O₃ supported Ni@TiOₓ granular catalyst.

### Example 2

A catalyst is prepared and reacts through the method in Example 1, and Example 2 differs from Example 1 in that in step (5), 0.06 part by mass of Ni(NO₃)₃·6H₂O is taken; and the mass percentage of TiOₓ is 10% based on a mass of the support, and the molecular formula is denoted as Ni1Ti6/Al₂O₃.

### Example 3

A catalyst is prepared and reacts through the method in Example 1, and Example 3 differs from Example 1 in that in step (5), 0.12 part by mass of Ni(NO₃)₃·6H₂O is taken; and a mass percentage of TiOₓ is 10% based on a mass of the support, and the molecular formula is denoted as Ni1Ti3/Al₂O₃.

### Example 4

A catalyst is prepared and reacts through the method in Example 1, and Example 4 differs from Example 1 in that in step (5), 0.36 part by mass of Ni(NO₃)₃·6H₂O is taken; and the mass percentage of TiOₓ is 10% based on the mass of the support, and the molecular formula is denoted as Ni1Ti1/Al₂O₃.

### Example 5

A catalyst is prepared and reacts through the method in Example 1, and Example 5 differs from Example 1 in that in step (5), 0 part by mass of Ni(NO₃)₃·6H₂O is taken; and the mass percentage of TiOₓ is 10% based on the mass of the support, and the molecular formula is denoted as TiOₓ/Al₂O₃.

### Example 6

Step (1): 0.09 part by mass of Ni(NO₃)₃·6H₂O is dissolved in 1 mL of deionized water;
Step (2): 1 part by mass of Al₂O₃ is impregnated in the above solution for ultrasonic treatment for 0.5-1 hour, then naturally dried at room temperature for 12 hours, and then completely dried at 80-100° C.;
Step (3): the solid obtained in step (2) is calcined under air atmosphere at 600° C. for 3 hours and reduced at 600° C. for 1 hour, to obtain the Al₂O₃ supported Ni catalyst, wherein the molecular formula is denoted as Ni/Al₂O₃;
Step (4): the prepared Al₂O₃ supported Ni catalyst is mechanically mixed with the Al₂O₃ supported TiOₓ catalyst prepared through the method in Example 5, and the mixture denoted as Ni/Al+TiOₓ/Al is pressed into pallets and sieved to granular catalysts at 20-40 meshes; and
Step (5): the granular catalyst is put into a fixed bed reactor, and reaction gas is introduced for reaction, wherein the molar ratio of hydrogen to propane is 1:1, the mass space velocity of propane is 3 h⁻¹, and the balance gas is nitrogen.

### Example 7

A catalyst is prepared and reacts through the method in Example 1, and Example 7 differs from Example 1 in that in step (1), 2.294 parts by mass of aluminum tri-sec-butoxide (ATSB) and 0.212 part by mass of tetrabutyl titanate (TTB) are taken; and the mass percentage of TiOₓ is 5% based on a mass of the support.

### Example 8

A catalyst is prepared and reacts through the method in Example 1, and Example 8 differs from Example 1 in that in step (1), 1.932 parts by mass of aluminum tri-sec-butoxide (ATSB) and 0.85 part by mass of tetrabutyl titanate (TTB) are taken; and the mass percentage of TiOx is 20% based on a mass of the support.

### Example 9

A catalyst is prepared and reacts through the method in Example 1, and Example 9 differs from Example 1 in that the calcination temperature in step (7) is 500° C.

### Example 10

A catalyst is prepared and reacts through the method in Example 1, and Example 10 differs from Example 1 in that the calcination time in step (7) is 4 hours.

### Example 11

A catalyst is prepared and reacts through the method in Example 1, and Example 11 differs from Example 1 in that the reducing temperature in step (3) is 400° C.

### Example 12

A catalyst is prepared and reacts through the method in Example 1, and Example 12 differs from Example 1 in that the reducing temperature in step (3) is 500° C.

### Example 13

A catalyst is prepared and reacts through the method in Example 1, and Example 13 differs from Example 1 in that the reducing temperature in step (3) is 550° C.

### Example 14

A catalyst is prepared and reacts through the method in Example 1, and Example 14 differs from Example 1 in that the reducing temperature in step (3) is 700° C.

### Example 15

A catalyst is prepared and reacts through the method in Example 1, and Example 15 differs from Example 1 in that the reducing time in step (3) is 2 hours.

### Example 16

Step (1): 0.526 part by mass of Cr(NO₃)₃·9H₂O is dissolved in 1 mL of deionized water;
Step (2): 1 part by mass of Al₂O₃ is impregnated in the above solution for ultrasonic treatment for 0.5-1 hour, then naturally dried at a room temperature for 12 hours, and then completely dried at 80-100° C.;
Step (3): the solid obtained in step (2) is calcined under air atmosphere at 600° C. for 3 hours and then reduced at 600° C. for 1 hour, to obtain the Al₂O₃ supported CrOₓ catalyst, wherein the molecular formula is denoted as CrOₓ/Al₂O₃; and a mass percentage of CrOₓ is 20% based on a mass of support;
Step (4): the prepared catalyst is pressed into pallets and sieved to granular catalysts at 20-40 meshes;
Step (5): the granular catalyst is put into a fixed bed reactor, and reaction gas is introduced for reaction, wherein the molar ratio of hydrogen to propane is 1:1, the mass space velocity of propane is 3 h⁻¹, and the balance gas is nitrogen.

Catalytic performance for propane dehydrogenation reactions of the catalysts prepared in the above examples are tested, activity of the catalysts is represented by propane conversion, propylene selectivity, propylene yield and deactivation rate, and it is discussed below with reference to calculated results:

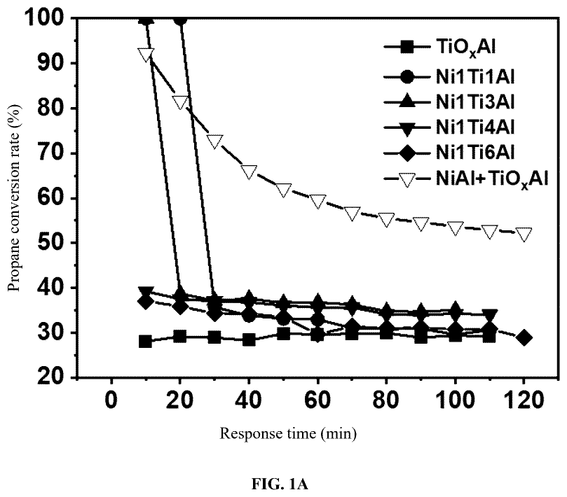

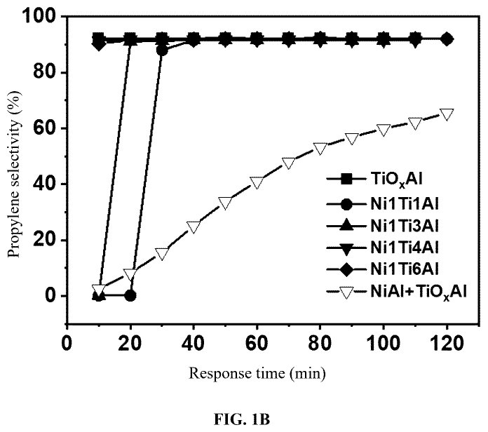

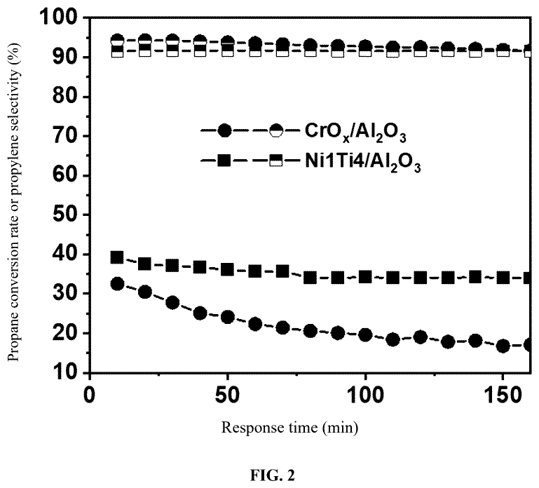

Catalytic performance for propane dehydrogenation reactions of the different NimTin/Al₂O₃ catalysts corresponding to Examples 1-6 are tested, the results are shown in FIGS. 1A-B, wherein FIG. 1A is a curve of propane conversion over time, and FIG. 1B is a curve of propylene selectivity over time. Compared with Ni/Al₂O₃ and TiOₓ/Al₂O₃ catalysts, NimTin/Al₂O₃, m:n=1:(1-6) have higher propylene selectivity and dehydrogenation activity. Wherein, the Ni1Ti4/Al₂O₃ catalyst has significantly improved catalytic activity while keeping high selectivity towards propylene, the initial conversion rate is about 40%, the propylene yield can reach about 16.70 mmol·g_{cat}⁻¹·h⁻¹ based on the mass of the catalyst, and according to a diagram showing comparison between catalytic performance of the catalysts corresponding to Examples 1 and 16 in FIG. 2, high activity comparable to that of industrial CrOₓ/Al₂O₃ catalyst is achieved. Meanwhile, due to more prominent C—C bond activation capacity of Ni sites, the mechanically mixed Ni/Al+TiOₓ/Al catalyst of Ni/Al₂O₃ and TiOₓ/Al₂O₃ shows a catalytic behavior pattern similar to that of the Ni/Al₂O₃ catalyst at the initial stage of reaction, but its catalytic behavior pattern is gradually transformed to that of the TiOₓ/Al₂O₃ catalyst at the later stage of reaction as the more reactive Ni sites are covered by deposited carbon, and the propylene yield is significantly improved. It is shown from the above results that metallic Ni and TiOₓ species in the mechanically mixed catalyst cannot effectively interact to form a Ni@TiOₓ core-shell structure due to difference in spatial distribution, rendering the metallic Ni sites exposed on a surface, and the significant difference in the catalytic performance further proved that TiOₓ shell serves as the active site for propane dehydrogenation on Ni@TiOₓ/Al₂O₃.

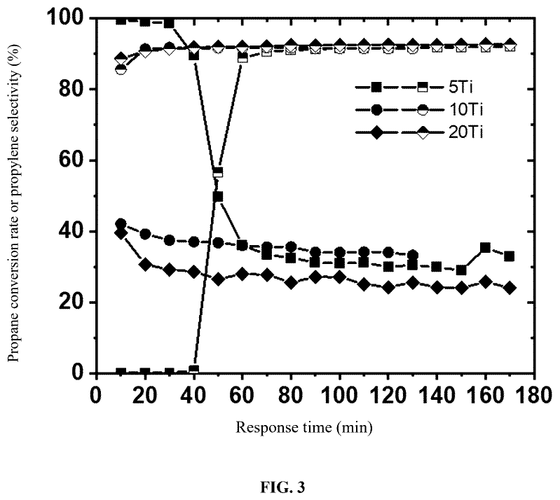

Examples 1, 7, and 8 are catalysts with different TiOₓ mass percentages (based on the mass of the support) and their catalytic performance for propane dehydrogenation are shown in FIG. 3. an induction period for producing methane is observed when the TiOₓ mass percentage is reduced to 5%, which may be caused by the fact that part of the Ni sites are exposed due to the correspondingly higher Ni/TiOₓ ratio. The catalytic performance is optimal when the TiOₓ content is 10%.

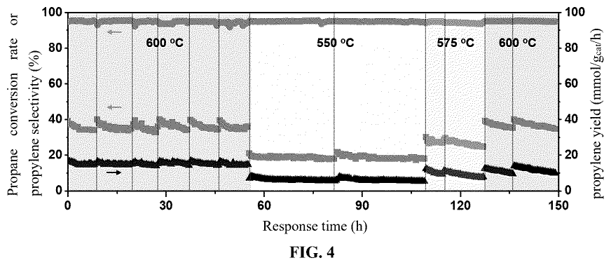

Long-term regeneration stability of the catalyst corresponding to Example 1 is further tested, results are shown in FIG. 4, it can be shown that the catalyst can completely restore to initial activity after being regenerated in successive dehydrogenation-regeneration cycle along with steady-state high selectivity towards propylene, achieving excellent stability, and deactivation rate constants corresponding to reaction temperatures of 550° C., 575° C., and 600° C. are 0.007 h⁻¹, 0.018 h⁻¹, and 0.073 h⁻¹ respectively, which are far lower than that of an industrial CrOₓ/Al₂O₃ catalyst under the same test conditions (deactivation rate constant corresponding to 600° C. is 0.32 h⁻¹).

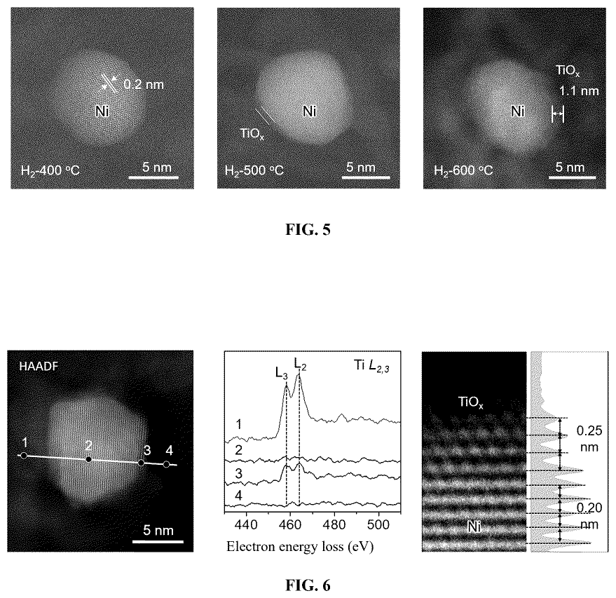

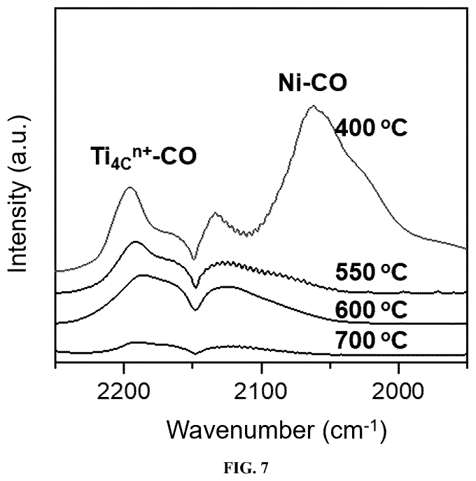

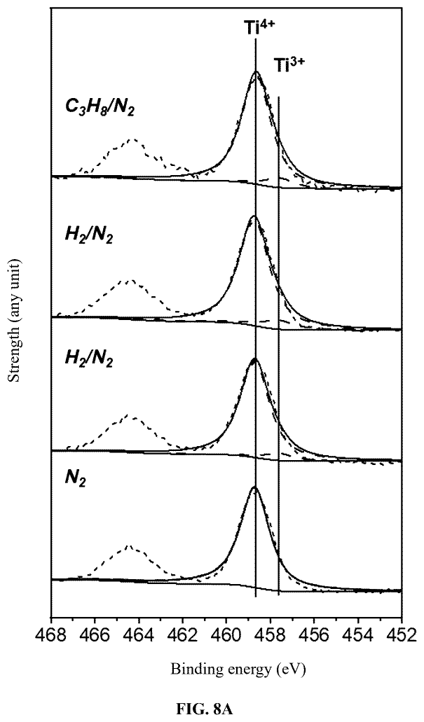

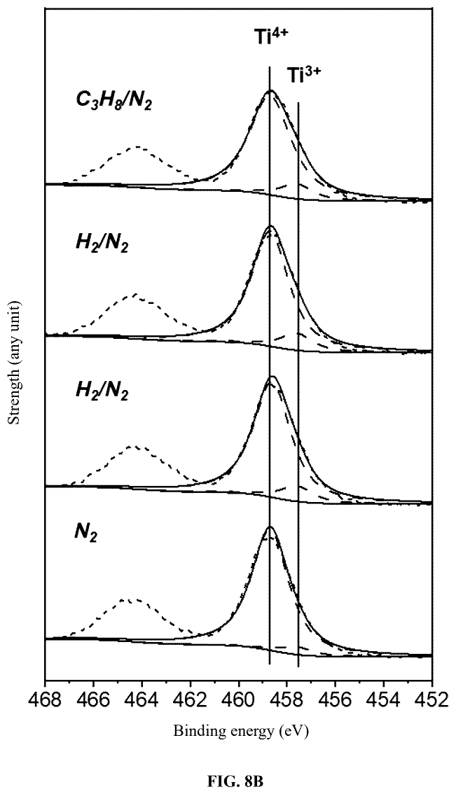

FIG. 5 is a spherical aberration-scanning transmission electron microscope images of Examples 11, 12, and 1 (Ni1Ti4/Al₂O₃ corresponds to H₂ reduction temperatures of 400° C., 500° C., and 600° C.) during in-situ reduction. FIG. 6 is the electron energy loss spectral line scanning profile corresponding to Example 1. Starting from 400° C. corresponding to Example 11, metallic Ni nanoparticles with an average diameter of about 6.8 nm can be observed, the formation of discrete and non-uniform TiOₓ overlayer is observed on surfaces of the Ni NPs when the reaction temperature rises to 500° C., and a uniform and relatively thicker (1-2 nm) TiOₓ overlayer is formed when the reaction temperature rises to 600° C., achieving complete coverage of metal Ni NPs (nano-particles). The results are consistent with CO adsorption infrared spectroscopy results of Examples 11, 13, 1, and 14 (Ni1Ti4/Al₂O₃ corresponds to H₂ reduction temperatures of 400° C., 550° C., 600° C., and 700° C.) during in-situ reduction in FIG. 7, the CO adsorption band belonging to metallic Ni at 2055 cm⁻¹ of the Ni1Ti4/Al₂O₃ catalyst is obvious when the reaction temperature is 400° C., the band gradually decreases until it disappears completely when the reducing temperature rises to 550° C. or above, indicating Ni sites exposed on the surface of the catalyst are gradually covered with the rise of the reducing temperature, proving the reverse encapsulation of Ni sites by TiOₓ overlayers induced by strong interaction. FIG. 6 specifically shows element distribution of Ni1Ti4/Al₂O₃ reduced at 600° C., it is observed that distances between Ni—Ni and Ti—Ti atoms are 0.20 nm and 0.25 nm respectively, and the latter corresponds to the compressed rutile titanium oxide (110) lattice spacing caused by the formation of Ni—TiOₓ interface during reduction. It is confirmed from line scanning profile of Ti L_{2,3} edge electron energy loss spectrum that Ti is distributed on the edge of NPs where Ni is absent.

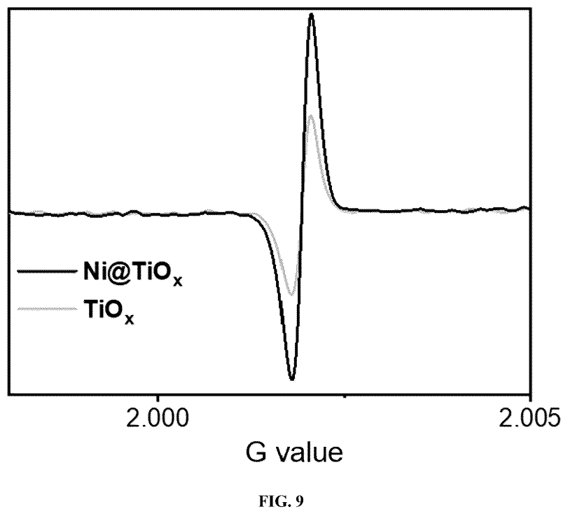

In-situ XPS spectral analysis is performed on the catalysts in Examples 1 and 5 to analyze chemical bonding and valence state distribution of surface Ti and O species changing over atmosphere, results are shown in FIGS. 8A-B, wherein FIG. 8A and FIG. 8B respectively correspond to the catalysts prepared in Examples 5 and 1. After H₂ reduction treatment at 600° C., compared with the TiOₓ/Al₂O₃ catalyst corresponding to Experimental example 5, the shape of dominant peak belonging to Ti⁴⁺ becomes more asymmetric for the Ni@TiOₓ/Al₂O₃ catalyst corresponding to Example 1 due to the emergence of the peak belonging to Ti³⁺ with lower bonding energy (457.5 eV) and it is shown from peak deconvolution results that the ratio of Ti³⁺/(Ti³⁺+Ti⁴⁺) is about twice that of TiOₓ/Al₂O₃, indicating the formation of more O vacancies. The results are consistent with EPR results of the catalysts in Examples 1 and 5 in FIG. 9, compared with the TiOₓ/Al₂O₃ catalyst corresponding to Experimental example 5, the signal intensity attributed to TiOₓ species with oxygen vacancies is significantly increased for the Ni@TiOₓ/Al₂O₃ catalyst corresponding to Experimental example 1, suggesting the concentration increase in oxygen vacancies and neighbor coordinatively unsaturated Ti sites in TiOₓ.

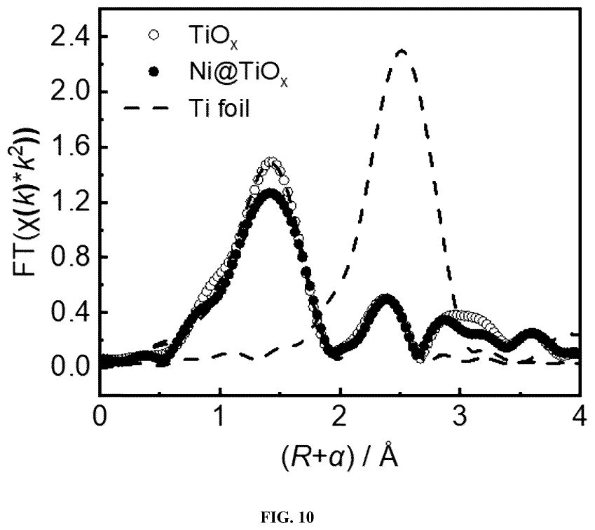

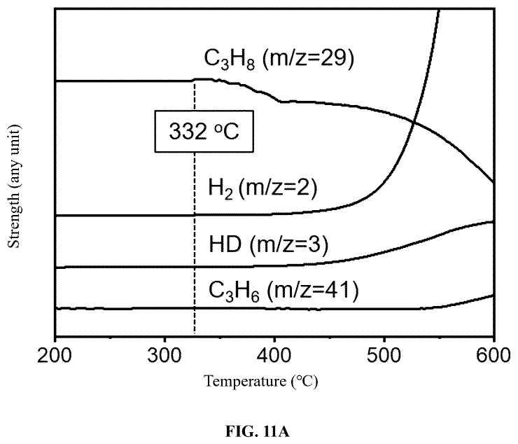

The Ti atomic coordination environment is obtained by further performing fitting analysis on Ti K-edge extended X ray absorption fine structures of the catalysts in Examples 1 and 5, as shown in FIG. 10, and compared with the TiOₓ/Al₂O₃ catalyst corresponding to Example 5, the Ti—O coordination number of the Ni@TiOₓ/Al₂O₃ catalyst corresponding to Example 1 decreases from 4.2 to 3.8, further providing experimental evidence for the concentration increase of the coordinatively unsaturated Ti sites. According to the above results, it is suggested that the higher catalytic activity of the Ni@TiOₓ/Al₂O₃ catalyst derives from the formation of the Ni@TiOₓ core-shell structure. The strong metal-oxide interaction triggers reverse encapsulation of metal Ni by TiOₓ overlayer during H₂ reduction treatment where intimate contact between the metal Ni and the TiOₓ overlayer during formation of this special “adhesion” structure facilitates hydrogen spillover, resulting in the formation of more O vacancies and corresponding coordinatively unsaturated Ti sites. The coordinatively unsaturated Ti_{4C} sites are active sites of propane dehydrogenation with higher C—H bond activation ability owing to lower C—H activation barrier, which greatly reduces an apparent reaction barrier to achieve higher propane dehydrogenation activity.

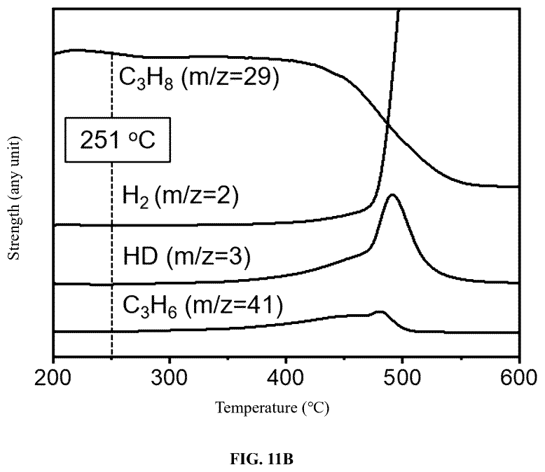

A propane-temperature programmed surface reaction test is performed on the catalysts in Examples 1 and 5 to represent the ability for activating C—H bonds of the catalytic active sites, results are shown in FIGS. 11A-B, wherein FIG. 11A and FIG. 11B respectively correspond to the catalysts prepared in Examples 5 and 1. In order to avoid H₂ signal interference from individual desorption of residual surface-adsorbed H species after H₂ reduction treatment, reduction treatment atmosphere is changed from H₂ to D₂, to ensure that H¹ derives from C—H cleavage of propane molecules and the HD signal is recorded as criteria of the initial C—H activation temperature, it can be shown that the dehydrogenation reaction activation temperature of TiOₓ/Al₂O₃ corresponding to Experimental example 5 with low concentration of oxygen vacancies and corresponding coordinatively unsaturated Ti sites is about 332° C., whereas the dehydrogenation reaction activation temperature of Ni@TiOₓ/Al₂O₃ corresponding to Experimental example 1 with high concentration of oxygen vacancies and corresponding coordinatively unsaturated Ti sites is reduced to about 251° C., proving that a C—H activation barrier corresponding to Ni@TiOₓ/Al₂O₃ is reduced, and propane dehydrogenation activity is improved.

Although the preferred examples of the present disclosure are described with reference to the drawings above, the present disclosure is not limited to the above specific implementations, and the above specific implementations are only schematic instead of restrictive. Those ordinarily skilled in the art may also make many forms of specific transformations without departing from the purpose of the present disclosure and the scope protected by the claims under inspiration of the present disclosure, and these transformations all belong to the protection scope of the present disclosure.

1. A supported TiOₓ core-shell catalyst, adopting Al₂O₃ as a support, wherein the Al₂O₃ support is loaded with a Ni@TiOₓ core-shell structure, the Ni@TiOₓ core-shell structure comprises a metal Ni core and a TiOₓ (1<x<2) shell; and a molecular formula of the catalyst is denoted as NimTin/Al₂O₃, wherein m:n=1:(1-6).

2. A preparation method of the supported TiOₓ core-shell catalyst according to claim 1, wherein a mass percentage of TiOₓ is 5%-15% based on a mass of the Al₂O₃ support.

3. The preparation method of the supported TiOₓ core-shell catalyst according to claim 1, wherein m:n=1:4.

4. The preparation method of the supported TiOₓ core-shell catalyst according to claim 1, comprising the following steps:
(1) adding aluminum alkoxide, an organotitanium compound, and a surfactant to isopropanol solvent and stirring them to be mixed well;
(2) dropwise adding dilute nitric acid to the mixed solution obtained in step (1) to be hydrolyzed completely;
(3) aging sol obtained in step (2) at a room temperature, and completely drying the sol under vacuum;
(4) calcining the solid obtained in step (3) step by step;
(5) impregnating the solid obtained in step (4) in Ni(NO₃)₃·6H₂O solution to be completely dried after being ultrasonically dispersed well; and
(6) calcining the solid obtained in step (5), and then reducing the solid at 400-700° C., to obtain the Al₂O₃ supported Ni@TiOₓ core-shell catalyst.

5. The supported TiOₓ core-shell catalyst according to claim 4, wherein in step (1), the aluminum alkoxide is one of aluminum tri-sec-butoxide (ATSB) and aluminum isopropoxide (Al(Opri)₃); the organotitanium compound is one of tetrabutyl titanate (TTB) and isopropyl titanate (TTP); the surfactant is one of cetyl trimethyl ammonium bromide (CTAB) and cetyltrimethylammonium chloride (CTAC); and the organic alcohol solvent is isopropanol or ethanol.

6. The supported TiOₓ core-shell catalyst according to claim 4, wherein in step (3), the vacuum drying is to dry in a vacuum oven at 60-80° C. for 18-24 hours.

7. The supported TiOₓ core-shell catalyst according to claim 4, wherein in step (1), in step (4), the calcining step by step is to firstly calcine at 200-300° C. for 2-3 hours, and then ramp up to 500-600° C. for calcining for 3-4 hours.

8. The supported TiOₓ core-shell catalyst according to claim 4, wherein in step (1), in step (6), the calcining temperature is 500-600° C., and calcining time is 2-4 hours; and the reducing time is 1-2 hours.

9. An application of the supported TiOₓ core-shell catalyst according to claim 1 in preparation of olefin from dehydrogenation of light alkane.

10. The application of the supported TiOₓ core-shell catalyst in preparation of olefin from dehydrogenation of light alkane according to claim 9, wherein the light alkane is propane, and the olefin is propylene.

11. The preparation method of the supported TiOₓ core-shell catalyst according to claim 2, comprising the following steps:
(1) adding aluminum alkoxide, an organotitanium compound, and a surfactant to isopropanol solvent and stirring them to be mixed well;
(2) dropwise adding dilute nitric acid to the mixed solution obtained in step (1) to be hydrolyzed completely;
(3) aging sol obtained in step (2) at a room temperature, and completely drying the sol under vacuum;
(4) calcining the solid obtained in step (3) step by step;
(5) impregnating the solid obtained in step (4) in Ni(NO₃)₃·6H₂O solution to be completely dried after being ultrasonically dispersed well; and
(6) calcining the solid obtained in step (5), and then reducing the solid at 400-700° C., to obtain the Al₂O₃ supported Ni@TiOₓ core-shell catalyst.

12. The preparation method of the supported TiOₓ core-shell catalyst according to claim 3, comprising the following steps:
(1) adding aluminum alkoxide, an organotitanium compound, and a surfactant to isopropanol solvent and stirring them to be mixed well;
(2) dropwise adding dilute nitric acid to the mixed solution obtained in step (1) to be hydrolyzed completely;
(3) aging sol obtained in step (2) at a room temperature, and completely drying the sol under vacuum;
(4) calcining the solid obtained in step (3) step by step;
(5) impregnating the solid obtained in step (4) in Ni(NO₃)₃·6H₂O solution to be completely dried after being ultrasonically dispersed well; and
(6) calcining the solid obtained in step (5), and then reducing the solid at 400-700° C., to obtain the Al₂O₃ supported Ni@TiOₓ core-shell catalyst.

13. The application of the supported TiOₓ core-shell catalyst according to claim 9, wherein a mass percentage of TiOₓ is 5%-15% based on a mass of the Al₂O₃ support.

14. The application of the supported TiOₓ core-shell catalyst according to claim 9, wherein m:n=1:4.
